(19) United States
(12) Patent Application Publication
(10) Pub. No.: US 2005/0171497 A1
(43) Pub. Date: Aug. 4, 2005

(54) Disposable underpants having absorbing pad

(76) Inventor: Wen-Lin Chiang, Taichung (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/768,047

(22) Filed: Feb. 2, 2004

(51) Int. Cl. A61F 13/15; A61F 13/20

(52) U.S. Cl. 604/385.01

(57) ABSTRACT

A disposable underpants includes a waist portion and two legs holes. An absorbent pad is stitched to a crotch region on an inside of the underpants and includes two sides, a front end and a rear end. The front end of the absorbent pad is located at a position of wearer's pubis and the rear end of the absorbent pad is located at a position of wearer's buttocks. A width of the rear end of the absorbent pad is larger than a width of the front end of the absorbent pad.

### FIELD OF THE INVENTION

The present invention relates to a disposable underpants including an absorbing pad attached to an inside of the underpants and covering an area from the pubis to the buttocks of the user.

### BACKGROUND OF THE INVENTION

Sanitary napkins are used during the period of female and attached to an inside of the underpants. The conventional sanitary napkin generally is an elongated article and includes two long sides and two short sides, and is made of absorbent material so as to absorb the body fluid. Leakage is always the main concern when carrying a sanitary napkin, abutment of the napkin with the body of the wearer decides the how serious that the risk of leakage may happen. Rearward leakage is a particular problem and normally occurs when the user lies down. Because the sanitary napkin is attached to the underpants by two adherent strips which cover only a very limited small area so that the sanitary napkin is easily deformed or shifted when a force exerts to it form the wearer's thighs when the wearer sits down for example. Wrinkles of the napkin could also result in leakage. There are too many situations that the wearers are in worry when a conventional sanitary napkin is attached to the underpants simply by two adherent strips.

The present invention intends to provide a disposable underpants that includes an absorbent pad integrally connected to an inside thereof and covering an area from the pubis to the buttocks of the wearer.

### SUMMARY OF THE INVENTION

The present invention relates to a disposable underpants comprises a waist portion and two legs holes. An absorbent pad is connected to a crotch region on an inside of the underpants and includes two sides, a front end and a rear end. The front end of the absorbent pad is located at a position of the wearer's pubis and the rear end of the absorbent pad is located at a position of the wearer's buttocks. A width of the rear end of the absorbent pad is larger than a width of the front end of the absorbent pad.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a front view of the underpants of the present invention;

FIG. 2 is a cross sectional view to show the underpants of the present invention, and

FIG. 3 shows a wearer wearing the underpants of the present invention.

### DETAILED DESCRIPTION OF THE PREFERRED EMBODIMENT

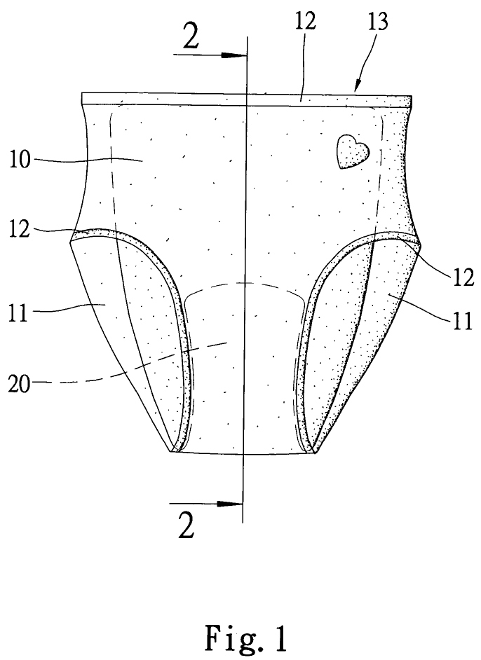

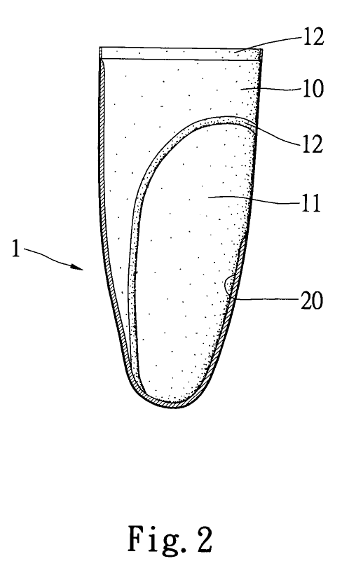

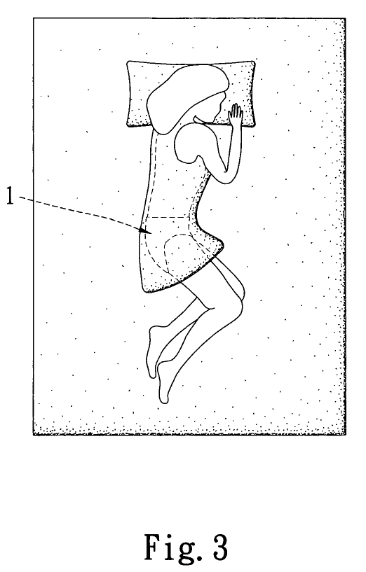

Referring to FIGS. 1 to 3, the disposable underpants 10 of the present invention is made of flexible material and comprises a waist portion 13 and two legs holes 11. Each of the two leg holes 11 and the waist portion 13 includes a flexible band 12 attached to a periphery thereof so that the underpants is matched to the wearer's body. An absorbent pad 20 is connected to a crotch region on an inside of the underpants 10 and including two sides, a front end and a rear end. The absorbent pad 20 can be stitched to the underpants 10 and the front end of the absorbent pad 20 is located at a position of wearer's pubis and the rear end of the absorbent pad 20 is located at a position of wearer's buttocks. A width of the rear end of the absorbent pad 20 is two times of a width of the front end of the absorbent pad 20. The rear end of the absorbent pad 20 reaches to the waist portion of the underpants 10 so as to have a large enough area to absorb the body fluid and to prevent the rearward leakage.

The underpants 10 of the present invention combines the underpants and the sanitary pad as one and the area that the absorbent pad 20 covers is large enough to prevent rearward leakage. The underpants 10 of the present invention is manufactured easily at lower cost and can be discarded after being used.

While we have shown and described the embodiment in accordance with the present invention, it should be clear to those skilled in the art that further embodiments may be made without departing from the scope of the present invention

1. A disposable underpants comprising:
a waist portion and two legs holes, an absorbent pad connected to a crotch region on an inside of the underpants and including two sides, a front end and a rear end, the front end of the absorbent pad being adapted to locate at a position of wearer's pubis and the rear end of the absorbent pad being adapted to locate at a position of wearer's buttocks, a width of the rear end of the absorbent pad being larger than a width of the front end of the absorbent pad.

2. The underpants as claimed in claim 1, wherein each of the two leg holes includes a flexible band attached to a periphery thereof.

3. The underpants as claimed in claim 1, wherein the width of the rear end of the absorbent pad is two times of the width of the front end of the absorbent pad.

4. The underpants as claimed in claim 1, wherein the absorbent pad is stitched to the underpants.

5. The underpants as claimed in claim 1, wherein the rear end of the absorbent pad reaches to the waist portion of the underpants.

6. The underpants as claimed in claim 1, wherein the waist portion includes a flexible band attached to a periphery thereof.
